(19) United States
(12) Patent Application Publication
(10) Pub. No.: US 2026/0338762 A1
(43) Pub. Date: Oct. 29, 2026

(54) DETECTION OF LIVER DISEASE

(71) Applicant: OWLSTONE MEDICAL LIMITED, Cambridge (GB)

(72) Inventors: Max ALLSWORTH, Cambridge (GB); Marc VAN DER SCHEE, Cambridge (GB); Giuseppe FERRANDINO, Cambridge (GB); Olga GANDELMAN, Cambridge (GB)

(21) Appl. No.: 19/118,333

(22) PCT Filed: Oct. 4, 2023
(86) PCT No.: PCT/GB2023/052566

§ 371 Date: Apr. 3, 2025

(30) Foreign Application Priority Data
Oct. 6, 2022 (GB) 2214670.8

(51) Int. Cl. G01N 33/50

(52) U.S. Cl. CPC G01N 33/50; G01N 2800/085; G01N 2800/52; G01N 2800/56; G01N 2800/60

(57) ABSTRACT

The invention relates to a method for detecting, staging, monitoring or prognosing a liver disease in a subject. The method comprises measuring the concentration of 2-pentanone, 4-methyl-1-pentene and/or 1-hexene, indole, dimethyl selenide, limonene, eucalyptol and (1-propylnonyl)benzene in a biological sample obtained from the subject.

### FIELD OF THE INVENTION

The invention relates to methods for the detection of a liver disease, such as non-alcoholic steatohepatitis (NASH) and related methods and kits.

### INTRODUCTION

Hepatic cirrhosis is scarring (fibrosis) of the liver which is typically caused by long-term liver damage. Cirrhosis can be classified into four stages: 1) steatosis, 2) scarring or fibrosis, 3) cirrhosis and 4) liver failure, advanced liver disease or hepatic failure. A patient may develop liver cirrhosis as a result of factors including, but not limited to, excessive alcohol consumption, viral hepatitis, autoimmune hepatitis etc.

Nonalcoholic Fatty Liver Disease (NAFLD) encompasses an entire histologic spectrum ranging from simple, benign hepatic steatosis to non-alcoholic steatohepatitis (NASH) characterized by lipid accumulation, inflammation, hepatocyte ballooning, and varying degrees of fibrosis. NASH may progress to cirrhosis or hepatocellular carcinoma (HCC). Other complications may include hepatic encephalopathy or liver failure. NASH, unlike non-alcoholic fatty liver disease (NAFLD), has the greatest potential to progress to cirrhosis, liver failure, and liver cancer. The prevalence of NAFLD is increasing and is linked to the increase in cases of obesity. Despite increasing awareness of obesity-related liver disease, the pathogenesis of NAFLD and NASH remains poorly understood.

Based on the prevalence of NAFLD, it is anticipated that NASH-induced cirrhosis will become the most common indication for liver transplantation in the future. Differentiating NASH from simple steatosis is important for the clinical management of NAFLD patients and to reduce mortality (Chen et al, Radiology. 2011 June; 259 (3): 749-756).

The sole test approved for NASH diagnosis is liver biopsy, an invasive procedure that can lead to complications. Surrogate methods lack adequate performance in early NASH stages and overall, this limits NASH early detection and makes it difficult to evaluate the efficacy of experimental drugs. Thus, there is a need for alternative diagnostic tests, in particular tests that can diagnose NASH and differentiate NASH from other stages of NAFLD, i.e., NAFL. Identification of both NAFLD and NASH non-invasively would help to significantly reduce the risk associated with diagnosis of these pathologies. Differentiating between NASH and NAFL allows for earlier lifestyle changes, medical interventions, cancer screening, and overall improved outcomes.

The use of exogenous volatile organic compound (EVOC®) probes for induced volatolomics—monitoring the metabolic processing of an exogenous compound by monitoring exhaled breath—to detect liver disease has been described in WO2019220145. The method described in WO2019220145 requires the administration of an exogenous probe to a subject.

Analysis of volatile organic compounds (VOCs) in exhaled breath represents an emerging diagnostic approach with the potential to develop functional, non-invasive tests for early detection based on the reduced hepatic function associated with chronic liver diseases, especially clearance and protein synthesis capacity. It has previously been shown that limonene, an exogenous volatile organic compound (EVOC) taken-up mainly through the diet, was found elevated in the breath of patients with cirrhosis and showed diagnostic potential in agreement with previous exploratory studies (Ferrandino G, Orf I, Smith R, Calcagno M, Thind A K, Debiram-Beecham I, et al. Breath Biopsy Assessment of Liver Disease Using an Exogenous Volatile Organic Compound-Toward Improved Detection of Liver Impairment. Clin Transl Gastroenterol 2020; 11(9)). This compound is metabolized in the liver by the enzymes CYP2C9 and CYP2C19 to trans-carveol and perillyl alcohol, respectively. In the cirrhotic liver, reduced activity of these enzymes impairs hepatic clearance, resulting in extended limonene half-life in the bloodstream, which in turn, raises its abundance in the breath. It has also been shown that limonene correlates with blood metrics that reflect hepatic clearance and protein synthesis capacity, indicating that a limonene breath test could be applied as functional means for chronic liver diseases detection. Nonetheless, combining multiple altered VOCs could improve test performances.

The method as described herein for cirrhosis detection, diagnostics, staging, monitoring and prognosis provides alternative tests for cirrhosis.

### SUMMARY OF THE INVENTION

The inventors have shown that impaired hepatic function shifts the spectrum of compounds detoxified by the liver. A subset of these can be measured in breath as VOCs and represent potential biomarkers. Therefore, the amount in breath represents liver function. Detecting each of the volatile compounds can increase diagnostic accuracy. The shift in the spectrum of compounds can be used as a proxy of liver function, which is affected in cirrhosis.

As such, the present inventors have developed a non-invasive test to detect and stage cirrhosis in a subject with or suspected of having liver disease for detection, diagnostics, staging, monitoring and prognosis (i.e., at risk of progression to a more advanced liver disease stage). For example, the test may be used to detect and stage non-alcoholic fatty liver disease (NAFLD) progressed to the stage of non-alcoholic steatohepatitis (NASH) in a subject with or suspected of having liver disease for detection, diagnostics, staging, monitoring and prognosis (i.e., at risk of progression to a more advanced liver disease stage).

In a first aspect, the invention relates to a method for detecting, staging, monitoring or prognosing a liver disease in a subject, the method comprising measuring the concentration of each of the following compounds in a biological sample obtained from the subject:
i) 2-pentanone,
ii) 4-methyl-1-pentene and/or 1-hexene,
iii) indole,
iv) dimethyl selenide,
v) limonene,
vi) eucalyptol and
vii) (1-propylnonyl)benzene.

In one embodiment, the method comprises:
(a) establishing a set of test subject values based on the concentration of each of compounds i) to vii) in the biological sample obtained from the subject; and
(b) comparing the set of test subject values to a set of reference values, wherein a difference in the set of test subject values and the set of reference values indicates a likelihood of liver disease. In one embodiment, the subject has not been administered with an exogenous substrate for an enzyme associated with a liver disease.

In one embodiment, the set of reference values are based on the concentration of each of compounds i) to vii) in a biological sample obtained from a subject that has been diagnosed with a liver disease.

In one embodiment, the set of reference values are based on the concentration of each of compounds i) to vii) obtained from a biological sample obtained from a healthy subject.

In one embodiment, the concentration of each of 2-pentanone, 4-methyl-1-pentene and/or 1-hexene, indole, limonene, eucalyptol and (1-propylnonyl)benzene in a biological sample obtained from the subject are elevated compared to the concentration of said compounds in a biological sample obtained from a healthy subject.

In one embodiment, the concentration of dimethyl selenide in a biological sample obtained from the subject is reduced compared to the concentration of indole in a biological sample obtained from a healthy subject.

In one embodiment, the biological sample is selected from breath, urine, blood, serum, and/or tissue.

In one embodiment, the biological sample is breath.

In one embodiment, the method comprises A) the subject inhaling purified air and B) the subject providing an exhaled breath sample, and wherein step A) is carried out before step B).

In one embodiment, the liver disease is a liver disease with cirrhosis.

In one embodiment, the liver disease is non-alcoholic steatohepatitis (NASH), such as NASH without fibrosis, NASH with fibrosis, NASH with hepatocellular carcinoma (HCC) or NASH with cirrhosis, for example decompensated cirrhosis.

In a second aspect, the invention relates to a method for detecting or prognosing early-stage cirrhosis, the method comprising measuring the concentration of each of the following compounds in a biological sample obtained from the subject:
i) 2-pentanone,
ii) 4-methyl-1-pentene and/or 1-hexene,
iii) indole,
iv) dimethyl selenide,
v) limonene,
vi) eucalyptol and
vii) (1-propylnonyl)benzene.

In one embodiment, the method the method comprises:
(a) establishing a set of test subject values based on the concentration of each of the following compounds i) to vii) in the biological sample obtained from the subject; and
(b) comparing the set of test subject values to a set of reference values, wherein a difference in the set of test subject values and the set of reference values indicates a likelihood of cirrhosis.

In a third aspect, the invention relates to a method for determining efficacy of a treatment in a subject diagnosed with a liver disease, comprising the steps of:
(a) obtaining a first biological sample from the patient before initiation of the treatment or therapy (or at a first time point after initiation of the treatment or therapy, or when the treatment or therapy is initiated);
(b) determining the concentration of each of compounds i) to vii) in the first sample;
(c) obtaining a second biological sample from the patient after initiation of the treatment or therapy (or at a second time point after initiation of the treatment or therapy);
(d) determining the concentration of each of compounds i) to vii) in the second sample; and
(e) comparing the concentration of each of compounds i) to vii) in the first sample with the concentration of each of compounds i) to vii) in the second sample.

In a fourth aspect, the invention relates to a method for detecting, staging, monitoring or prognosing liver disease in a subject, comprising measuring the concentration of an exogenous compound and/or metabolite thereof in a biological sample obtained from said subject; wherein the exogenous compound is a substrate for an enzyme, which substrate may be metabolised to the metabolite by said enzyme in vivo, and/or is a compound that may undergo oxidative degradation in vivo to form the metabolite; wherein the exogenous compound is a generally recognised as safe (GRAS) compound; and wherein the exogenous compound is selected from tryptophan, a C1-C20 alkylbenzene, organic selenium, inorganic selenium and/or an unsaturated fatty acid.

In a fifth aspect, the invention relates to a method for detecting, staging, monitoring or prognosing liver disease in a subject, comprising measuring the concentration of two or more exogenous compounds and/or metabolites thereof in a biological sample obtained from said subject; wherein each exogenous compound is a substrate for an enzyme, which substrate may be metabolised to the metabolite by said enzyme in vivo, and/or is a compound that may undergo oxidative degradation in vivo to form the metabolite; wherein each exogenous compound is a generally recognised as safe (GRAS) compound; and wherein the exogenous compounds are selected from:
i) one or more of eucalyptol and/or limonene; and
ii) one or more of 2-pentanone, tryptophan, a C1-C20 alkylbenzene, such as (1-propylnonyl)benzene, organic selenium, inorganic selenium and/or an unsaturated fatty acid.

In one embodiment, the method comprises measuring the concentration of each of the following exogenous compounds and/or metabolites thereof: I) eucalyptol, II) limonene, III) 2-pentanone, IV) tryptophan, V) a C1-C20 alkylbenzene, such as (1-propylnonyl)benzene, VI) organic selenium, VII) inorganic selenium and VIII) an unsaturated fatty acid.

In one embodiment, the metabolite of tryptophan comprises indole.

In one embodiment, the metabolite of a C1-C20 alkylbenzene, such as (1-propylnonyl)benzene, comprises benzoic acid and/or hippuric acid.

In one embodiment, the metabolite of organic selenium and/or inorganic selenium comprises dimethyl selenide.

In one embodiment, the metabolite of an unsaturated fatty acid comprises an alkene, for example a C2-C20 alkene, for example a C2-C10 alkene, for example a C4-C8 alkene, for example a C6 alkene, or for example 4-methyl-1-pentene and/or 1-hexene.

the metabolite of eucalyptol comprises 2-alpha-hydroxy-1,8-cineole and/or 3-alpha-hydroxy-1,8-cineole; and/or

In one embodiment, the metabolite of limonene comprises trans-carveol and/or perillyl alcohol.

In one embodiment, the metabolite of 2-pentanone comprises 2-pentanol, 3-hydroxy-2-pentanone and/or 2,3-pentanediol, for example 2-pentanol.

In a further aspect, the invention relates to the use of one or more exogenous compounds selected from tryptophan, a C1-C20 alkylbenzene, organic selenium, inorganic selenium and/or an unsaturated fatty acid in a method according to claim 15.

In a further aspect, the invention relates to the use of two or more exogenous compounds selected from i) one or more of eucalyptol and/or limonene and ii) one or more of 2-pentanone, tryptophan, a C1-C20 alkylbenzene, organic selenium, inorganic selenium and/or an unsaturated fatty acid, in a method according to claim 16.

In a further aspect, the invention relates to a kit for the detection, diagnosis, screening or prognosis of a liver disease or for determining efficacy of a treatment for liver disease comprising one or more exogenous compounds selected from tryptophan, a C1-C20 alkylbenzene, organic selenium, inorganic selenium and an unsaturated fatty acid and a device for capturing a biological sample from a patient.

In a further aspect, the invention relates to a kit for the detection, diagnosis, screening or prognosis of a liver disease or for determining efficacy of a treatment for liver disease comprising two or more exogenous compounds selected from:
i) one or more of eucalyptol and/or limonene; and
ii) one or more of tryptophan, a C1-C20 alkylbenzene, organic selenium, inorganic selenium and an unsaturated fatty acid and a device for capturing a biological sample from a patient;
   and a device for capturing a biological sample from a patient.

### BRIEF DESCRIPTION OF THE FIGURES

FIG. 1—Volcano plot of exhaled VOCs. The X axes represents the log 2 mean ratio fold-change of the relative abundance of each VOC between cirrhosis and healthy. The y-axis represents the p-value for each VOC. Compounds with fold change >2 and p-Value <0.05 have been highlighted in blue. Limonene and 2-pentanone were elevated in the breath of patients with cirrhosis, while dimethyl selenide was reduced, as expected.

FIG. 2—ROC plots of single top 4 VOCs. Top 4 ROC-plots for on-breath VOCs were built to explore their discriminatory performance. 2-pentanone, limonene and dimethyl selenide were found among them.

FIG. 3 Classification performance of combined VOCs. (A) ROC plot and confidence interval obtained for the training set; (B) ROC plot and confidence interval obtained for the test set; (C) Corresponding confusion matrix generated using the Youden index as threshold; (D) Improvements of classification performance by addition of VOCs to the model and (E) ROC plots showing improvement of classification performance as more features are added to the classification model up to the 7 top performing VOCs.

FIG. 4 Correlation of breath VOCs with blood metrics of liver function in cirrhosis subjects. (A) Correlation plot of identified VOCs and serum bilirubin, albumin, and INR. Blue indicates a negative and red a positive correlation. Circle size and intensity of the colour represent the magnitude of the correlation. (B) The CCA score plot using the first canonical variates of selected sets of VOCs and blood metrics of liver function. Each projected data point represents the combined information of breath VOCs and blood metrics of one cirrhotic patient. The CCA analysis revealed significant correlations with R² 0.767.

FIG. 5—VOCs alterations in relation to cirrhosis severity. Projected data points of the first 2 components of a PCA calculated using breath variables with an BH adjusted p-value <0.1. PC1 explains 10.1% and PC2 4.4% of variance and show separation of cirrhotic patients based on their CP score. About 50% of the patients with CP=5 cluster with healthy.

### DETAILED DESCRIPTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The present invention provides methods of detecting, staging, screening, diagnosing, monitoring or prognosing a liver disease. Advantageously the present inventors have developed methods of detecting a liver disease which may be non-invasive or minimally invasive as the method is performed on a biological sample.

### Untargeted Methods

In the first aspect, the invention relates to a method for detecting, staging, monitoring or prognosing a liver disease in a subject, the method comprising measuring the concentration of each of compounds i) to vii) in a biological sample obtained from the subject.

In the second aspect, the invention relates to a method for detecting or prognosing early stage non-alcoholic steatohepatitis (NASH), the method comprising measuring the concentration of each compounds i) to vii) in a biological sample obtained from the subject.

In the third aspect, the invention relates to a method for determining efficacy of a treatment in a subject diagnosed with a liver disease, comprising the steps of:
(a) obtaining a first biological sample from the patient before initiation of the treatment or therapy (or at a first time point after initiation of the treatment or therapy, or when the treatment or therapy is initiated);
(b) determining the concentration of each of compounds i) to vii) in the first sample;
(c) obtaining a second biological sample from the patient after initiation of the treatment or therapy (or at a second time point after initiation of the treatment or therapy);
(d) determining the concentration of each of compounds i) to vii) in the second sample; and
(e) comparing the concentration of each of compounds i) to vii) in the first sample with the concentration of each of compounds i) to vii) in the second sample.

The methods of the first, second and third aspects of the invention suitably do not involve administrating the subject with an exogenous substance. Such methods may be described as an “untargeted method” (because an exogenous substrate is not administered to target a disease-associated enzyme of interest). As such, these methods do not involve administrating the subject with an exogenous substrate for an enzyme, for example an enzyme associated with liver disease.

Preferably, the subject has not been administered with an exogenous substrate for an enzyme associated with a liver disease, for example a NASH-associated enzyme.

An “exogenous substrate” is any compound that can be administered to a subject that is metabolised by an enzyme within the subject. An exogenous substrate refers to a chemical compound that is recognized by the enzyme of interest and for which the enzyme catalyzes conversion of the substrate into a different chemical compound which is referred to herein as a “metabolite”. An “exogenous substrate” is a xenobiotic, i.e., a substance that is foreign to the subject's body and which is specifically and selectively metabolised by the enzyme.

The liver disease may be any suitable liver disease.

The liver disease may be a liver disease with cirrhosis.

The liver disease may be a non-alcoholic fatty liver disease (NAFLD).

The liver disease may be non-alcoholic steatohepatitis (NASH), such as NASH without fibrosis, NASH with fibrosis, NASH with hepatocellular carcinoma (HCC) or NASH with cirrhosis, for example decompensated cirrhosis.

NAFLD is a term that refers to a range of conditions which are caused by a build-up of fat in the liver. The term encompasses a disease spectrum which includes a mild benign form of the disease where there is a build-up of fat in the liver, referred to as steatosis or NAFL. This disease state can progress to a more severe form known as NASH wherein the liver becomes inflamed, this stage may also be referred to herein as NASH without fibrosis. NASH can then progress to NASH with fibrosis, where persistent inflammation causes scar tissue around the liver and nearby blood vessels to form. The most severe form of the disease is NASH with cirrhosis which can occur after long term inflammation, resulting in shrinkage and scarring of the liver. NASH with cirrhosis causes permanent damage to the liver and can lead to liver failure and development of liver cancer (hepatocellular carcinoma (HCC)).

The severity of NAFLD can be described among three stages. Stage 1 is characterized by simple fatty liver (i.e. NAFL or hepatic steatosis). Fat begins to accumulate in individual cells but liver function is normal. There are usually no symptoms and patients may not realize they have the condition. Although the fat deposits are considered harmless, it is important to prevent the disease from progressing to the next stage.

Stage 2 is often referred to as NASH. NASH is a more aggressive form of the condition, where the liver has become inflamed. Inflammation is the body's healing response to damage or injury and, in this case, is a sign that liver cells have become damaged. A person with NASH may have a dull or aching pain felt in the top right of their abdomen (over the lower right side of their ribs). NASH can occur with or without fibrosis.

Stage 3 is often characterized by cirrhosis. At this most severe stage, bands of scar tissue and clumps of liver cells develop. The liver shrinks and becomes lumpy which is known as cirrhosis. Cirrhosis progresses slowly gradually causing the liver to stop functioning. The damage caused by cirrhosis is irreversible and the patient may experience signs of liver failure. Cirrhosis tends to occur after the age of 50, usually after years of liver inflammation associated with the early stages of the disease. People with cirrhosis of the liver caused by NAFLD often also have type 2 diabetes.

The term “cirrhosis,” “liver cirrhosis” or “hepatic cirrhosis” refers to a condition in which the liver does not function properly due to long-term damage. This damage is characterized by the replacement of normal liver tissue by scar tissue (i.e. fibrosis). The disease generally develops slowly over months or years, often with no symptoms. Eventually, excessive scar formation will result in loss of liver function.

The stage may be early-stage NASH. Early-stage NASH is different from NAFL and is characterised by steatosis, inflammation, high hepatic fat and hepatocellular injury.

The term “prognosis” refers to the forecast or likely outcome of a disease. As used herein, it refers to the probable outcome of liver disease, including whether the disease (e.g. NASH) will respond to treatment or mitigation efforts and/or the likelihood that the disease will progress.

The term progression as used herein may refer to an advancement of the disease state. The term regression as used herein may refer to a decrease of the severity of the disease state. When the disease is monitored, this can result in detecting progression or regression.

Regression may be due to health style changes or therapeutic intervention, for example using a treatment as described herein, including a treatment in clinical drug trials.

The methods require measuring the concentration of each of compounds i) to vii) in a biological sample from a subject. The term biological sample may be used interchangeably with the term biological matrix. The biological sample or matrix may be selected from breath, urine, blood, serum, and/or tissue. The biological sample may be a tissue sample such as adipose tissue, liver, brain, bone marrow, muscle or hair. The biological sample may be a sample of bodily fluid. Methods are well known in the art for obtaining bodily fluid samples. The bodily fluid sample may be a sample of blood, urine or exhaled breath. The sample of blood may comprise one or more of blood plasma, red blood cells, white blood cells, platelets. The blood sample may comprise any combination of blood plasma, red blood cells, white blood cells, platelets.

The biological sample may be exhaled breath.

Where the bodily fluid sample is a sample of exhaled breath, the breath sample can include air exhaled from one or more different parts of the subject's body (e.g. nostrils, pharynx, trachea, bronchioles, alveoli etc.). For the collection of a breath sample and methods of measurement, the device and methods described in WO2017/187120 or WO2017/187141 (both publications are hereby incorporated by reference) can be used.

In embodiments wherein the biological sample is a sample of exhaled breath, this may be obtained by collecting exhaled air from the subject, for example by requesting the subject to exhale air into a gas-sampling container, such as a bag, a bottle or any other suitable gas-sampling product. Preferably the gas-sampling container resists gas permeation both into and out of the bag and/or is chemically inert, thereby assuring sample integrity. Exhaled breath may also be collected using a breath collector apparatus. Preferably, collection of a sample of exhaled breath is performed in a minimally invasive or a non-invasive manner.

The determination of the amount of VOCs in a sample of exhaled breath from a subject may be performed by the use of at least one technique including, but not limited to, Gas-Chromatography (GC), Gas-Chromatography-lined Mass Spectrometry (GC/MS), Liquid Chromatography-tandem mass spectrometry (LC/MS), Ion Mobility Spectrometry/Mass Spectrometry (IMS/MS), Proton Transfer Reaction Mass-Spectrometry (PTR-MS), Electronic Nose device, quartz crystal microbalance or chemically sensitive sensors.

The amount of VOCs in a sample of exhaled breath from a subject may be determined using thermal desorption-gas chromatography-time of flight-mass spectrometry (GC-tof-MS). In certain embodiments, breath of the subject is collected in an inert bag, then the content of the bag is transported under standardised conditions onto desorption tubes and VOCs are analyzed by thermally desorbing the content of the tube and then separated by capillary gas chromatography. Then volatile organic peaks are detected with MS and identified using for example a library, such as the National Institute of Standards and Technology. Thermal desorption may be performed at the GC inlet at a temperature of, e.g., about 200-350 °C. In all chromatography, separation occurs when the sample mixture is introduced (injected) into a mobile phase. Gas chromatography (GC) typically uses an inert gas such as helium as the mobile phase. GC/MS allows for the separation, identification and/or quantification of individual components from a biological sample. MS methods which may be used with the present invention include, but are not limited to, electron ionization, electrospray ionization, glow discharge, field desorption (FD), fast atom bombardment (FAB), thermospray, desorption/ionization on silicon (DIOS), Direct Analysis in Real Time (DART), atmospheric pressure chemical ionization (APCI), secondary ion mass spectrometry (SIMS), spark ionization and thermal ionization (TIMS). Matrix assisted laser desorption ionization time-of-flight mass spectrometry (MALDI-TOF-MS) is an example of a mass spectroscopy method which may be used to determine VOCs from a sample of exhaled breath from a subject.

The methods may comprise collecting different selected exhaled breath samples, or fractions thereof, on a single breath sample capture device, the method comprising the steps of:
(a) collecting a first exhaled breath sample by contacting the sample with a capture device comprising an adsorbent material;
(b) collecting a second exhaled breath sample by contacting the second sample with said capture device, wherein the first and second exhaled breath samples are caused to be captured on the capture device in a spatially separated manner.

Thus, the invention also relates to a method comprising:
i) collecting a first exhaled breath sample by contacting the sample with a capture device comprising an adsorbent material;
ii) collecting a second exhaled breath sample by contacting the second sample with said capture device, wherein the first and second exhaled breath samples are caused to be captured on the capture device in a spatially separated manner; and
iii) establishing a set of test subject values based on the concentration of each of the following compounds in the first and second exhaled breath samples: i) 2-pentanone, ii) 4-methyl-1-pentene and/or 1-hexene, iii) indole, iv) dimethyl selenide, v) limonene, vi) eucalyptol and vii) (1-propylnonyl)benzene; and
iv) comparing the set of test subject values to a set of reference values, wherein a difference in the set of test subject values and the set of reference values indicates a likelihood of liver disease.

The capture device may comprise an adsorbent material in the form of a porous polymeric resin. Suitable adsorbent materials include Tenax® resins and Carbograph® materials. Tenax® is a porous polymeric resin based on a 2,6-diphenyl-p-propylene oxide monomer. Carbograph® materials are graphitized carbon blacks. In one embodiment, the material is Tenax GR, which comprises a mixture of Tenax® TA and 30% graphite. One Carbograph® adsorbent is Carbograph 5TD. The capture device may comprise both Tenax GR and Carbograph 5TD. The capture device is conveniently a sorbent tube. These are hollow metal cylinders, typically of standard dimensions (3½ inches in length with a ¼ inch internal diameter) packed with a suitable adsorbent material.

Where the biological sample is exhaled breath, the method may comprise the steps of A) the subject inhaling purified air and B) the subject providing an exhaled breath sample, and wherein step A) is carried out before step B). Advantageously, the subject inhaling purified air prior to a sample of exhaled breath being taken (for analysis) may minimise environmental contribution to the signal.

As such, the methods may comprise the steps of:
A) administering purified air to the subject by inhalation;
B) collecting a sample of exhaled breath from the subject; and
C) measuring the concentration of each of compounds i) to vii) in the sample of exhaled breath obtained from the subject,
   wherein step A) is carried out before step B).

The biological sample may be obtained at any time point.

The biological sample may be obtained before or after treatment or therapy. The biological sample may be obtained at any time point before or after the treatment or therapy, such as about 10 minutes, about 30 minutes, about 1 hour, about 2 hours, about 3 hours, about 4 hours, about 5 hours, about 6 hours, about 7 hours, about 8 hours, about 10 hours, about 12 hours, about 15 hours, about 18 hours, about 20 hours, about 22 hours, about 1 day, about 2 days, about 3 days, about 4 days, about 5 days, about 6 days, about 1 week, about 2 weeks, about 3 weeks, about 1 month, about 2 months, about 3 months, about 4 months, about 5 months, about 6 months, about 1 year, about 2 years, about 3 years, about 5 years or longer before or after the start of the treatment or therapy. The time point may also be earlier or later.

The methods may comprise:
(a) establishing a set of test subject values based on the concentration of each compound i) to vii) in the biological sample obtained from the subject; and
(b) comparing the set of test subject values to a set of reference subject values,
   wherein a difference in the set of test subject values and the set of reference subject values indicates a likelihood of liver disease.

The concentration of each of compounds i) to vii) can be measured using methods known in the art. The concentration as used herein means the content or mass of each of compounds i) to vii) in the biological sample as expressed, for example in grams/litre (g/l).

As used herein, the “set” of test or reference subject values includes the concentration of each of i) 2-pentanone, ii) 4-methyl-1-pentene and/or 1-hexene, iii) indole, iv) dimethyl selenide, v) limonene, vi) eucalyptol, vii) (1-propylnonyl)benzene, in a biological sample from a test subject or reference subject, respectively. As such, each set of test subject values comprises a value for the concentration of each of compounds i) to vii) in a biological sample obtained from a test subject. Each set of reference subject values comprises a value for the concentration of each of compounds i) to vii) in a biological sample obtained from a reference subject.

Each test and/or reference subject value in the set of test or reference subject values may be a discreet value or may be a range of values. For example, the test subject value corresponding to i) 2-pentanone may be a discreet value or may be a range of values, etc.

As used herein, “reference subject value”, “reference subject values” etc. means a value, or values, determined by performing the testing method on one or more reference subjects. “Reference subject values” are used interchangeably with “baseline subject values” herein.

The methods of the present invention involve determining the concentration of each of compounds i) to vii) in a biological sample obtained from the test subject and then comparing the concentration to a set of reference subject values.

The reference subject values are representative of the concentration of each of compounds i) to vii) in a healthy subject and/or a subject that has been diagnosed with a liver disease.

The reference subject may be a healthy subject. As used herein, “healthy subject” is defined as a subject, i.e., a human, that does not have a liver disease of interest. For example, a heathy subject may be a healthy person or a non-liver disease subject not suffering from or destined to develop a liver disease.

As such, the set of reference values may be based on the concentration of each of compounds i) to vii) obtained from a biological sample obtained from a healthy subject.

The reference subject may be a subject that has been diagnosed with a liver disease.

The reference subject may be a subject that has been diagnosed with cirrhosis.

The reference subject may be a subject that has been diagnosed with NASH.

The reference subject may be a subject that has been diagnosed with non-alcoholic fatty liver (NAFL).

The reference subject may be a subject that has progressed to cirrhosis or HCC.

As such, the set of reference values may be based on the concentration of each of compounds i) to vii) in a biological sample obtained from a subject that has been diagnosed with a liver disease, for example, NASH, for example NAFL, or for example cirrhosis or HCC.

Reference subject values can be determined by measuring the concentration of each of compounds i) to vii) in a sufficiently large number of samples obtained from normal, healthy control subjects to obtain a pre-determined reference or threshold value. Reference value(s) can also be determined by measuring the concentration of each of compounds i) to vii) in a sample from a patient prior to treatment.

Variation of in the level of each of compounds i) to vii) from the reference subject values (either up or down) indicates that the patient has a liver disease, an increased risk of a liver disease and/or an increased risk of long-term mortality. For example, the concentration of each of compounds i) to vii) in the set of test subject values may individually be elevated or reduced compared to the reference values.

The concentration of each of i) 2-pentanone, ii) 4-methyl-1-pentene and/or 1-hexene, iii) indole, v) limonene, vi) eucalyptol, and vii) (1-propylnonyl)benzene, has been shown to be elevated in patients suffering from a liver disease, such as NASH, when compared to healthy individuals.

As such, the concentration one or more of i) 2-pentanone, ii) 4-methyl-1-pentene and/or 1-hexene, iii) indole, v) limonene, vi) eucalyptol, and vii) (1-propylnonyl)benzene, in a biological sample obtained from the subject may be elevated compared to the concentration of said compounds in a biological sample obtained from a healthy subject. The concentration two, three, four, five or each of i) 2-pentanone, ii) 4-methyl-1-pentene and/or 1-hexene, iii) indole, v) limonene, vi) eucalyptol, and vii) (1-propylnonyl)benzene, may be elevated compared to the concentration of said compounds in a biological sample obtained from a healthy subject.

The concentration of iv) dimethyl selenide has been shown to be reduced in patients suffering from a liver disease, such as NASH, when compared to healthy individuals.

As such, the concentration of iv) dimethyl selenide in a biological sample obtained from the subject may be reduced compared to the concentration of iv) dimethyl selenide in a biological sample obtained from a healthy subject.

When the liver disease is a NAFLD, the reference subject values may correspond to values calculated from NAFL subjects. The presence of test subject values for one, more or each of i) 2-pentanone, ii) 4-methyl-1-pentene and/or 1-hexene, iii) indole, v) limonene, vi) eucalyptol, and vii) (1-propylnonyl)benzene, at quantities greater than their respective range of healthy subject values may indicate a substantial likelihood of a NASH disease state in the test subject. The presence of test subject values for iv) dimethyl selenide at quantities lower than their respective range of healthy subject values may indicate a substantial likelihood of a NASH disease state in the test subject.

When the liver disease is a NAFLD, the reference subject values may correspond to values calculated from NAFL subjects. The presence of test subject values for one, more or each of i) 2-pentanone, ii) 4-methyl-1-pentene and/or 1-hexene, iii) indole, v) limonene, vi) eucalyptol, and vii) (1-propylnonyl)benzene at quantities greater than their respective range of healthy subject values may indicate a substantial likelihood of a NASH disease state in the test subject. The presence of test subject values for iv) dimethyl selenide at quantities lower than their respective range of healthy subject values may indicate a substantial likelihood of a NASH disease state in the test subject.

A difference in the set of test subject values and the set of reference subject values indicates a likelihood of liver disease. A “likelihood of a liver disease” means that the probability that the liver disease exists in the subject specimen is about 50% or more, for example 60%, 70%, 80% or 90%.

When the set of reference subject values are obtained from a healthy subject, an increased concentration of one, more or each of i) 2-pentanone, ii) 4-methyl-1-pentene and/or 1-hexene, iii) indole, v) limonene, vi) eucalyptol, and vii) (1-propylnonyl)benzene, e.g., 5%, 10%, 20%, 30%, 40%, 50% or more, in the test subject values compared to the reference subject values may indicate a diagnosis of a liver disease, such as NASH, or a risk that the subject will develop a liver disease, such as NASH.

When the set of reference subject values are obtained from a healthy subject, a decreased concentration of iv) dimethyl selenide, e.g. 5%, 10%, 20%, 30%, 40%, 50% or more, in the test subject values compared to the reference subject values may indicate a diagnosis of a liver disease, such as NASH, or a risk that the subject will develop a liver disease, such as NASH.

When the set of reference subject values are obtained from a subject that has been diagnosed with a liver disease, a concentration of one, more or each of i) 2-pentanone, ii) 4-methyl-1-pentene and/or 1-hexene, iii) indole, v) limonene, vi) eucalyptol, and vii) (1-propylnonyl)benzene, e.g., 5%, 10%, 20%, 30%, 40%, 50% or more, that is the same, substantially the same or increased in the test subject values compared to the reference subject values may indicate a diagnosis of a liver disease, such as NASH, or a risk that the subject will develop a liver disease, such as NASH.

When the set of reference subject values are obtained from a subject that has been diagnosed with a liver disease, a concentration of iv) dimethyl selenide, e.g., 5%, 10%, 20%, 30%, 40%, 50% or more, that is the same, substantially the same or increased in the test subject values compared to the reference subject values may indicate a diagnosis of a liver disease, such as NASH, or a risk that the subject will develop a liver disease, such as NASH.

When an appropriate reference is indicative of a subject being free of a liver disease a detectable difference (e.g., a statistically significant difference) between the set of test subject values and the appropriate reference may be indicative of NASH in the subject.

When an appropriate reference is indicative of a liver disease, a lack of a detectable difference (e.g., lack of a statistically significant difference) between the test subject values and the appropriate reference may be indicative of NASH in the subject.

Thus, the methods may include diagnosing the subject as having a likelihood or increased risk of a liver disease if the level of one or more of compounds i) to vii) is different from the level of a healthy reference subject value. Additionally or alternatively, the methods may include diagnosing the subject as having a likelihood or increased risk of a liver disease if the level of one or more of compounds i) to vii) is the same, substantially the same or different from the level of a reference subject value, wherein the reference subject value is obtained from a subject that has been diagnosed with a liver disease.

Thus, any of the methods as described herein may further comprise the steps of:
a) comparing the amount of each of compounds i) to vii) in a biological sample with a set of reference subject values, said reference subject values representing a known diagnosis, prognosis and/or monitoring status of liver disease, such as NASH;
b) finding a deviation or no deviation of the amount of one or more of compounds i) to vii) from said reference values; and
c) attributing said finding of deviation or no deviation to a particular diagnosis, prognosis and/or monitoring status of a liver disease, such as NASH, in the subject.

The term “deviation of the amount” refers either to elevated or reduced amounts of one or more of compounds i) to vii) in a biological sample from a subject compared to the reference subject values. By “elevated amounts” we mean that the amount of said one or more of compounds i) to vii) in a biological sample from a subject is statistically higher than the corresponding reference subject value. By “reduced amounts” we mean that the amount of one or more of compounds i) to vii) in a biological sample from a subject is statistically lower than the corresponding reference subject value. The amount may be considered to be statistically higher or lower if its value differs from a predetermined threshold value. This threshold value can, for example, be the median of the amount of the compound determined in a biological sample from a population of healthy subjects.

The term “no deviation of the amount” refers to similar or unchanged amounts of one or more of compounds i) to vii) in a sample of exhaled breath from a subject compared to the corresponding reference subject value. By “similar or unchanged level” is meant that the difference of the amount of said one or more of compounds i) to vii) in a biological sample from the subject compared to the corresponding reference subject value is not statistically significant. Preferably, the reference subject value is obtained in samples of exhaled breath obtained from one or more subjects of the same species and the same sex and age group as the subject in which a liver disease is to be determined, prognosed or monitored. Alternatively, the reference subject value may be a previous value for the amount of one or more of compounds i) to vii) obtained in a sample of exhaled breath from a specific subject. This kind of reference value may be used if the method is to be used for monitoring the liver disease, e.g., over time, or to monitor the response of a subject to a particular treatment.

The method may also comprise determining a risk score of the subject based on the concentration each of compounds i) to vii) in the sample and using the risk score to provide a prognosis for the subject, wherein the risk score is indicative of said prognosis.

The algorithm used to calculate a risk assessment score in a method disclosed herein may group the concentration values of each of compounds i) to vii), and the risk score can be derived from any algorithm known in the art. The algorithms are sets of rules for describing the risk assessment of a liver disease. The rule set may be defined exclusively algebraically but may also include alternative or multiple decision points requiring domain-specific knowledge, expert interpretation or other clinical indicators. Many algorithms that can provide different risk assessments can be developed using concentration profiles of a suitable substrate and/or metabolite. For example, the risk scores of an individual may be generated using a Cox proportional hazard model. An individual's prognostic categorization can also be determined by using a statistical model or a machine learning algorithm, which computes the probability of recurrence based on the individual's concentration of each of compounds i) to vii).

Based on the determination of a risk, individuals can be partitioned into risk groups (e.g., tertiles or quartiles) based on a selected value of the risk score, where all individuals with values in a given range can be classified as belonging to a particular risk group. Thus, the values chosen will define risk groups of patients with respectively greater or lesser risk. Risk groups can further be classified on different ranges of mortality, for example, on 6 month, 1-year, 2-year, 3-year, 4-year, 5-year, 10-year, 25-year mortality. Risk groups can further be classified on different ranges of events associated with a liver disease. For example, when the liver disease is NASH, the risk groups can be classified on different ranges of events associated with NASH, which can include, but is not limited, likelihood of progression to NASH with fibrosis or NASH with cirrhosis.

The methods of the invention may include a step of administering a suitable treatment to treat liver disease following a diagnosis that the subject has a liver disease or is at risk of developing a liver disease. Thus, the invention also provides a method for treating liver disease in a subject, comprising the steps of: (a) establishing a set of test subject values based on the concentration of each of the following compounds in a biological sample obtained from the subject: i) 2-pentanone, ii) 4-methyl-1-pentene and/or 1-hexene, iii) indole, iv) dimethyl selenide, v) limonene, vi) eucalyptol, and vii) (1-propylnonyl)benzene; (b) comparing the set of test subject values to a set of reference values, wherein a difference in the set of test subject values and the set of reference values indicates a likelihood of liver disease and (c) treating the subject.

It will be appreciated that the step of treating the subject should only be performed when the difference in the set of test subject values and the set of reference values indicates a likelihood of liver disease, i.e., when the subject is diagnosed as having a liver disease or likely to have a liver disease.

Any suitable treatment can be selected. In particular, treatment may be selected from one or more of gastric bypass surgery, and/or a drug-based treatment comprising the administration of at least one drug selected from statins, incretin analogues, metformin, rimonabant, thiazolidinediones, and orlistat.

An aspect of the invention relates to a method for determining efficacy of a treatment or therapy in a subject diagnosed with a liver disease, comprising the steps of:
(a) obtaining a first biological sample from the patient before initiation of the treatment or therapy (or at a first time point after initiation of the treatment or therapy, or when the treatment or therapy is initiated);
(b) determining the concentration of each of compounds i) to vii) in the first sample;
(c) obtaining a second biological sample from the patient after initiation of the treatment or therapy (or at a second time point after initiation of the treatment or therapy);
(d) determining the concentration of each of compounds i) to vii) in the second sample; and
(e) comparing the concentration of each of compounds i) to vii) in the first sample with the concentration of each of compounds i) to vii) in the second sample.

The second biological sample may be taken at any suitable time point after initiation of the treatment or therapy. For example, the second biological sample may be taken hours, days or weeks after the initiation of the treatment or therapy. A further biological sample, such as a third, fourth, fifth, etc. biological sample, may be taken at a different time point to the second biological sample. It will be understood by the skilled person that taking two or more biological samples after initiation of the treatment or therapy may enable monitoring of the efficacy of a treatment or therapy over time.

Alternatively or additionally, the efficacy of a treatment or therapy may be monitored in real-time. For example, the efficacy of a treatment or therapy may be measured in real-time by selected ion flow tube mass spectrometry (SiFT-MS).

Depending on the compound, if the concentration increases or decreases compared to the concentration of the compound obtained in the first sample, the therapy is considered to be effective. An effective treatment or therapy may be continued, or discontinued if the patient's condition has improved and is no longer in need of treatment. An ineffective treatment may be altered or modified, or replaced with other treatment.

The treatment may comprise surgery or at least one drug selected from statins, incretin analogues, metformin, rimonabant, thiazolidinediones, and orlistat.

In order to determine the efficacy of treatment multiple samples may be obtained at various times points. As such the method may comprise analysing a first biological sample obtained from said subject at a first time point, and then analysing one or more additional biological samples obtained from said subject at one or more additional time points or ratios thereof.

Thus, the method may also comprise the step of administering a treatment.

When the liver disease is NASH, said treatment may be gastric bypass surgery, and/or a drug-based treatment comprising the administration of at least one drug selected from statins, incretin analogues, metformin, rimonabant, thiazolidinediones, and orlistat. Treatments of NASH are known in the art, se Ganguli et al Hepat Med. 2019; 11:159-178.

The technology described herein may be associated with a programmable machine designed to perform a sequence of arithmetic or logical operations as provided by the methods described herein. For example, some embodiments of the technology are associated with (e.g., implemented in) computer software and/or computer hardware. In one aspect, the technology relates to a computer comprising a form of memory, an element for performing arithmetic and logical operations, and a processing element (e.g., a microprocessor) for executing a series of instructions (e.g., a method as provided herein) to read, manipulate, and store data. Therefore, certain embodiments employ processes involving data stored in or transferred through one or more computer systems or other processing systems. Embodiments also relate to apparatus for performing these operations. This apparatus can be specially constructed for the required purposes, or it can be a general-purpose computer (or a group of computers) selectively activated or reconfigured by a computer program and/or data structure stored in the computer. In some embodiments, a group of processors performs some or all of the recited analytical operations collaboratively (e.g., via a network or cloud computing) and/or in parallel.

In some embodiments, a microprocessor is part of a system for determining the concentration of each of compounds i) to vii); generating standard curves; determining a specificity and/or sensitivity of an assay or marker; calculating an ROC curve; sequence analysis; all as described herein or is known in the art. The amount of each of compounds i) to vii) can be determined by abundance, measured per mole or millimole.

The concentration of each of compounds i) to vii) can be determined by assays known to the skilled person and described herein, including measurements using an optical signal or other measurement known to one of skill.

In some embodiments, a microprocessor or computer uses an algorithm to measure the concentration of each of compounds i) to vii). The algorithm can include a mathematical interaction between a marker measurement or a mathematical transform of a marker measurement. The mathematical interaction and/or mathematical transform can be presented in a linear, nonlinear, discontinuous or discrete manner.

In some embodiments, a software or hardware component receives the results of multiple assays and determines a single value result to report to a user that indicates a liver disease risk based on the results of the multiple assays. Related embodiments calculate a risk factor based on a mathematical combination (e.g., a weighted combination, a linear combination) of the results from multiple assays as described elsewhere herein.

### Targeted Methods

In a fourth aspect, the invention relates to a method for detecting, staging, monitoring or prognosing liver disease in a subject, comprising measuring the concentration of an exogenous compound and/or metabolite thereof in a biological sample obtained from said subject; wherein the exogenous compound is a substrate for an enzyme, which substrate may be metabolised to the metabolite by said enzyme in vivo, and/or is a compound that may undergo oxidative degradation in vivo to form the metabolite; wherein the exogenous compound is a generally recognised as safe (GRAS) compound; and wherein the exogenous compound is selected from tryptophan, a C1-C20 alkylbenzene, organic selenium, inorganic selenium and/or an unsaturated fatty acid.

In a fifth aspect the invention relates to a method for detecting, staging, monitoring or prognosing liver disease in a subject, comprising measuring the concentration of two or more exogenous compounds and/or metabolites thereof in a biological sample obtained from said subject; wherein each exogenous compound is a substrate for an enzyme, which substrate may be metabolised to the metabolite by said enzyme in vivo, and/or is a compound that may undergo oxidative degradation in vivo to form the metabolite; wherein each exogenous compound is a generally recognised as safe (GRAS) compound; and wherein the exogenous compounds are selected from:
i) one or more of eucalyptol and/or limonene; and
ii) one or more of 2-pentanone, tryptophan, a C1-C20 alkylbenzene, such as (1-propylnonyl)benzene, organic selenium, inorganic selenium and/or an unsaturated fatty acid.

The method of the fifth aspect of the invention comprises measuring the concentration of two or more exogenous compounds and/or measuring the concentration of two metabolites of said compounds in a biological sample obtained from a subject. The method is therefore a multiplex method enabling assessment of multiple enzymatic activities simultaneously in the same sample(s), such as the same breath sample(s). This enables a more accurate diagnosis due to multiple parameters that are assessed.

Suitable features of the fourth and fifth aspects of the invention are as described above in relation to the first, second and third aspects of the present invention. For example, suitable biological samples for the fourth and fifth aspects of the invention are as described above in relation to the first, second and third aspects of the present invention.

The method of the fourth, fifth, sixth and seventh aspects are based on administration of an exogenous substrate to a subject. Such methods may be described as an “targeted method” (because an exogenous substrate is administered to target either a disease-associated enzyme of interest or an oxidative degradation pathway that is associated with a disease of interest). This is in contrast to the first, second and third aspects of the invention wherein no exogenous substance is administered to a subject.

Thus, the methods according to fourth and fifth aspects of the invention may comprise a step of administering an exogenous compound, or at least two exogenous compounds as appropriate, as described herein. The exogenous compound may be a substrate for an enzyme. As defined above, an “exogenous substrate” is any compound that can be administered to a subject that is metabolised by an enzyme within the subject. An exogenous substrate refers to a chemical compound that is recognized by the enzyme of interest and for which the enzyme catalyzes conversion of the substrate into a different chemical compound which is referred to herein as a “metabolite”. The exogenous compound may be a compound that may undergo oxidative degradation in vivo. The compound may undergo oxidative degradation due to oxidative stress. The compounds used in the methods of the fourth and fifth aspect of the invention are exogenous substances, i.e. are xenobiotic. The term xenobiotic refers to a substance that is foreign to the subject's body. Preferably, the exogenous substance converted into a metabolite by the enzyme or by oxidative degradation is also a xenobiotic, that is it does not normally occur in the subject's body.

The exogenous compound is a generally recognised as safe (GRAS) compound.

The exogenous compound may be selectively metabolised by an enzyme within the subject. The enzyme is an enzyme whose activity or expression are downregulated in a liver disease, such as NASH, or whose activity or expression are upregulated in a liver disease, such as NASH, that is in patients that present with a liver disease, such as NASH, compared to healthy subjects. For example, activity or expression are upregulated or downregulated in liver tissue in liver disease, such as NASH patients, compared to healthy subjects. Thus, the changes in expression and/or activity of the enzyme are indicative of a liver disease, such as NASH.

For example, when the liver disease is NASH, the gene encoding for the enzyme may be differentially expressed in NASH tissue compared to non-NASH tissue. For example, the enzyme may be expressed at a higher level in NASH tissue compared to non-NASH tissue or at a lower level in NASH tissue compared to non-NASH tissue. In another embodiment, the enzyme may be differentially active in NASH tissue compared to non-NASH tissue. For example, the enzyme may be modified such that the activity of the enzyme is higher or lower in NASH tissue compared to the activity in non-NASH tissue. Gene expression can be measured by techniques known in the art, for example by mRNA quantification or measuring cDNA. The activity of an enzyme can be measured by evaluating its metabolic activity, that is the enzyme's capacity to metabolise a substrate.

Non-NASH tissue may refer for example to healthy tissue or to NAFLD tissue which has not progressed to NASH for example tissue from a subject with NAFL also referred to as steatosis. The tissue may be from a specific organ, e.g., liver, lung, colon, breast, prostate etc. In one embodiment, the tissue is liver tissue.

The methods of the fourth and fifth aspects of the invention may include an additional step of identifying a suitable enzyme whose activity or expression are downregulated in NASH or whose activity or expression are upregulated in NASH compared to non-NASH tissue. The methods of the invention may include a further step of identifying a substrate of the enzyme and optionally the metabolite produced due to the enzymatic action.

Thus, methods described herein in relation to the fourth and fifth aspects of the invention may indirectly measure the activity of enzymes that are directly associated with a liver disease state in a non-invasive or minimally invasive way by measuring the activity of the enzymes via the metabolism of substrates in a biological sample. Due to the association between the enzyme and its ability in breaking down a substrate and the liver disease state, a diagnosis or prognosis can be made as to the patient's disease state. On that basis, a suitable treatment can be selected. Suitable treatments are as defined herein.

In one embodiment, the methods of the fourth and fifth aspects of the invention include a step of administering a suitable treatment to treat liver disease, such as NASH, following a diagnosis that the subject has a liver disease, such as NASH, or is at risk of developing a liver disease, such as NASH. Thus, the methods may comprise the step of treating the subject.

Metabolism and transformation of a compound by one or more enzyme and/or by oxidative degradation leads to the generation of a breakdown product, that is a metabolic product, i.e., a metabolite. When the compound is a substrate for an enzyme, soon after provision of a substrate to the subject, the substrate is excreted into biological matrices such as breath, urine, blood at high levels and clearance of the substrate from said biological matrices occurs as a consequence of biotransformation of the substrate by the action of one or more enzymes (washout of the reactant). For example, the kinetic profile of the clearance of the substrate from breath may be used as a readout of the enzyme activity responsible for biotransformation of said substrate.

In addition, metabolism of a specific substrate through one or more enzyme leads to production of enzyme-specific metabolic products. In this case metabolic products are excreted into biological matrices over time, starting at low levels and increasing over time due to biotransformation of the substrate by the enzyme. Measurement of such a metabolic product can be applied as a probe for assessing the metabolic phenotype of the enzyme or enzymes responsible for the production of said product.

As explained further herein, the wash-out curves for certain metabolites are different between liver diseased and non-liver diseased tissue, such as NASH and non-NASH tissue, depending on whether the enzyme that metabolises the substrate is overexpressed or downregulated in liver diseased tissue, such as NASH tissue.

The exogenous compounds may be specific for an enzyme such that the substrate is selectively metabolised by the enzyme. The exogenous compound may therefore be any substrate that is suitable for detecting the enzyme activity. The exogenous compounds may be specific for a particular oxidative degradation pathway. The exogenous compounds may therefore be any compound that is suitable for detecting the oxidative degradation pathway. For example, the compound may be an unsaturated fatty acid and the oxidative degradation pathway may comprise lipid peroxidation (for example due to oxidative stress).

In an embodiment, the compound and/or its metabolite is a VOC that is secreted in biological matrices, preferably a VOC that is secreted into biological matrices at high proportions. Generally, VOCs are defined as organic chemical compounds whose composition makes it possible for them to evaporate under normal indoor atmospheric conditions of temperature and pressure. Since the volatility of a compound is generally higher the lower its boiling point temperature, the volatility of organic compounds is sometimes defined and classified by their boiling points. Volatile compounds are compounds that are secreted by the human body into gas fluids, including for example breath, skin emanations and others. Optionally the compound and/or metabolite is a VOC that can be measured in a biological matrix without the use of any labels, such as isotope labels.

Each compound may be a VOC and the concentration of the exhaled VOC substrate in breath may be measured.

Thus, the methods of the fourth and fifth aspects use an exogenous volatile organic compound (EVOC) as tracers of specific in vivo liver-specific metabolic activities. EVOCs can be volatile compounds that, administered to a subject through various routes, undergo metabolism and distribution in the body and are excreted via breath. Additionally, when the exogenous compound is a substrate for an enzyme, metabolism of EVOCs by liver-specific enzymes can lead to production of other volatile compounds that can also be detected in breath.

In one embodiment, each compound is a VOC and its metabolite is not a VOC. In this embodiment, the concentration of the substrate in breath is measured. In another embodiment, each compound is not a VOC and its metabolite is a VOC. In this case, the concentration of the metabolite in breath is measured. In another embodiment, the compound is a VOC and its metabolite is a VOC. In this case, the concentration of the compound and/or the metabolite in breath is measured.

If the compound is a VOC, it may be labelled or it may not be labelled.

The VOCs that are measured according to the methods of the fourth and fifth aspects may not be naturally occurring/produced by the subject and excreted into a biological matrix. This ensures that any readings are not contaminated by endogenous VOCs that are naturally produced and can be found in biological matrices.

In one embodiment, each compound may be a naturally occurring compound (but that is not endogenously produced), for example a food compound. This has the advantage that it can be provided to a subject without the occurrence of side effects. In one embodiment, each compound does not have any therapeutic benefit. In one embodiment, the compound is not a non-naturally occurring compound.

The compounds are each GRAS compounds, for example GRAS compounds that are a VOC. “GRAS” is an acronym for the phrase Generally Recognized As Safe. Under sections 201(s) and 409 of the Federal Food, Drug, and Cosmetic Act, any substance that is intentionally added to food is a food additive, that is subject to premarket review and approval by FDA, unless the substance is generally recognized, among qualified experts, as having been adequately shown to be safe under the conditions of its intended use, or unless the use of the substance is otherwise excepted from the definition of a food additive. For example, the GRAS compound can be a naturally occurring compound. For example, the GRAS compound can be selected from a food or food additive. In one embodiment, the GRAS compound is a vitamin, phenolic flavoring agent, natural oil, alcohol, amino acid or antioxidant. In one embodiment, the GRAS compound is a plant extract. In one embodiment, the GRAS compound is a plant substance primarily used for flavoring, coloring or preserving food. In one embodiment, the GRAS compound is an aliphatic or aromatic terpene hydrocarbon or a terpenoid.

In one embodiment, the or each compound is not a VOC and its metabolite is not a VOC. In such an embodiment, the or each compound may be a labelled reactant and the labelled reactant and/or labelled metabolite can be measured in breath. The label may be an isotope label, for example 12C, 13C, 14C, 2H, 14N or 18O.

In an embodiment, the or each compound and/or metabolite is a VOC and the compound is not labelled. Therefore, no labelling is required as the compound and/or metabolite can be measured in a biological matrix without the use of any labels.

The compound may be a substrate for an enzyme. An enzyme may be an alcohol dehydrogenase. A substate may be an alcohol and/or a ketone, for example a ketone. Alcohol metabolism is a well-characterized biological process that is dominated by the alcohol dehydrogenase (ADH) and aldehyde dehydrogenase (ALDH) families. Alcohol dehydrogenases catalyse the oxidation of primary and secondary alcohols to the corresponding aldehyde or ketone. Alterations in alcohol metabolism processes in response to human NASH progression have been investigated and the activity and expression of Alcohol dehydrogenase enzymes has been studied (Li, H., Toth, E. & Cherrington, N. J. Alcohol Metabolism in the Progression of Human Nonalcoholic Steatohepatitis. Toxicol Sci 164, 428-438, (2018)).

The alcohol dehydrogenase may be an aa-alcohol dehydrogenase. The alcohol dehydrogenase may be ALDH4A1, ADH1A, ADH1B, and ADH4.

An enzyme may be an alcohol dehydrogenase and a substrate may be a ketone, for example 2-pentanone. A metabolite may be 2-pentanol, 3-hydroxy-2-pentanone and/or 2,3-pentanediol, for example 2-pentanol. In diseased liver tissue, such as NASH tissue, reduced ADH activity may impair hepatic clearance of ketones, such as 2-pentanone, resulting in extended ketone, such as 2-pentanone, half-life in the bloodstream, which in turn, may raise its abundance in exhaled breath. As such, an enzyme may be an alcohol dehydrogenase and a metabolite may be 2-pentanol, 3-hydroxy-2-pentanone and/or 2,3-pentanediol, for example 2-pentanol.

An enzyme may be an alcohol dehydrogenase and a substrate may be 2-pentanone. An enzyme may be an alcohol dehydrogenase and a substrate may be 2-pentanone and a metabolite may be 2-pentanol, 3-hydroxy-2-pentanone and/or 2,3-pentanediol.

An enzyme may be a cytochrome P450 (CYP450) enzyme. The CYP450 enzyme family is responsible for metabolism of most drugs and lipophilic xenobiotics and are therefore of great importance for clinical pharmacology. Although several different families of CYP450 enzymes are present in the human body, the enzymes belonging to 1-, 2-, and 3-families are involved in the metabolism of the great majority of administered therapeutic drugs.

The CYP450 enzyme may be selected from families 1, 2 or 3. For example, the CYP450 enzyme is selected from CYP1A1, CYP1A2, CYP1 B1, CYP2, CYP2A6, CYP2A7, CYP2A13, CYP2B6, CYP2C8, CYP2C9, CYP2C18, CYP2C19, CYP2D6, CYP2E1, CYP2F1, CYP2J2, CYP2R1, CYP2S1, CYP2U1, CYP2W1, CYP3, CYP3A4, CYP3A5, CYP3A7 or CYP3A43. In one embodiment, the enzyme is CYP2C19, CYP2C9 and/or CYP3A4.

The CYP450 enzyme may be CYP2C9 and/or CYP2C19.

The CYP450 enzyme may be CYP3A4.

When an enzyme is a CYP450, a substrate may be a terpene and/or terpenoid. In diseased liver tissue, such as in a cirrhotic liver, reduced activity of CYP450 enzymes may impair hepatic clearance of terpenes and/or terpenoids, resulting in extended terpene and/or terpenoid half-life in the bloodstream, which in turn, may raise their abundance in exhaled breath.

A substrate may be limonene.

A substrate may be eucalyptol.

An enzyme may be CYP2C9 and/or CYP2C19 and a substrate may be limonene. Limonene is known to be metabolized by the enzymes CYP2C9 and CYP2C19 to trans-carveol and perillyl alcohol (Miyazawa M, Shindo M, Shimada T., Metabolism of (+)- and (−)-limonenes to respective carveols and perillyl alcohols by CYP2C9 and CYP2C19 in human liver microsomes. Drug Metab Dispos 2002; 30(5):602-607). As such, a metabolite may be trans-carveol and/or perillyl alcohol. An enzyme may be CYP2C9 and/or CYP2C19 and a metabolite may be trans-carveol and/or perillyl alcohol.

An enzyme may be CYP2C9 and/or CYP2C19 and a substrate may be limonene and a metabolite may be trans-carveol and/or perillyl alcohol.

An enzyme may be CYP3A4 and a substrate may be eucalyptol. Eucalyptol may be metabolised by the enzyme CYP3A4 to 2-alpha-hydroxy-1,8-cineole and/or 3-alpha-hydroxy-1,8-cineole. As such, a metabolite may be 2-alpha-hydroxy-1,8-cineole and/or 3-alpha-hydroxy-1,8-cineole,

An enzyme may be CYP3A4 and a substrate may be eucalyptol and a metabolite may be 2-alpha-hydroxy-1,8-cineole and/or 3-alpha-hydroxy-1,8-cineole.

An enzyme may be tryptophanase. A substrate may be tryptophan. A metabolite may be indole. It is known that indole is generated by the catabolism of tryptophan (and other substrates), which is typically mediated by gut bacteria (Stavropoulos G, van Munster K, Ferrandino G, Sauca M, Ponsioen C, van Schooten F J, et al. Liver Impairment—The Potential Application of Volatile Organic Compounds in Hepatology. Metabolites 2021; 11(9)). Impaired hepatic clearance may explain elevated levels of indole in cirrhosis. An enzyme may be tryptophanase and a substrate may be tryptophan. An enzyme may be tryptophanase and a metabolite may be indole.

An enzyme may be tryptophanase and a substrate may be tryptophan and a metabolite may be indole.

An enzyme may be an acyl CoA dehydrogenase, an enoyl CoA hydratase, a 3-hydroxyacyl-CoA dehydrogenase, a β-ketothiolase, an alcohol dehydrogenase, an aldehyde dehydrogenase, and/or a glycine N-acyltransferase. A substrate may be a C10-C20 alkylbenzene, such as (1-propylnonyl)benzene. Alkylbenzenes with a long alkyl group, such as (1-propylnonyl)benzene may be oxidized to the corresponding carboxylic acid and undergo β-oxidation to form benzoic acid, which is then converted to hippuric acid. This metabolic pathway was found downregulated in subjects with cirrhosis (Saltzman A, Caraway W T. Cinnamic acid as a test substance in the evaluation of liver function. J Clin Invest 1953; 32(8):711-719). Enzymes involved in this pathway may be an acyl CoA dehydrogenase, an enoyl CoA hydratase, a 3-hydroxyacyl-CoA dehydrogenase, a β-ketothiolase, an alcohol dehydrogenase, an aldehyde dehydrogenase, and/or a glycine N-acyltransferase. An enzyme may be an acyl CoA dehydrogenase, an enoyl CoA hydratase, a 3-hydroxyacyl-CoA dehydrogenase, a β-ketothiolase, an alcohol dehydrogenase, an aldehyde dehydrogenase, and/or a glycine N-acyltransferase and a substrate may be a C10-C20 alkylbenzene, such as (1-propylnonyl)benzene. An enzyme may be be acyl CoA dehydrogenase, enoyl CoA hydratase, 3-hydroxyacyl-CoA dehydrogenase, β-ketothiolase, alcohol dehydrogenase, aldehyde dehydrogenase, glycine N-acyltransferase and a metabolite may be benzoic acid and/or hippuric acid.

An enzyme may be an acyl CoA dehydrogenase, an enoyl CoA hydratase, a 3-hydroxyacyl-CoA dehydrogenase, a β-ketothiolase, an alcohol dehydrogenase, an aldehyde dehydrogenase, and/or glycine N-acyltransferase and a substrate may be (1-propylnonyl)benzene and a metabolite may be benzoic acid and/or hippuric acid.

An enzyme may be an S-adenosylmethionine: thioether-S-methyltransferase. A substrate may be organic and/or inorganic selenium. A metabolite may be dimethyl selenide. Dimethyl selenide is one of the excretion products of selenium metabolism (Burk R F, Hill K E. Regulation of Selenium Metabolism and Transport. Annu Rev Nutr 2015; 35:109-134). Impaired organic and inorganic selenium biotransformation may explain downregulation of dimethyl selenide in the breath of cirrhotic patients. An enzyme may be an S-adenosylmethionine: thioether-S-methyltransferase and a substrate may be organic and/or inorganic selenium. An enzyme may be an S-adenosylmethionine: thioether-S-methyltransferase and a metabolite may be dimethyl selenide.

An enzyme may be an S-adenosylmethionine: thioether-S-methyltransferase and a substrate may be organic and/or inorganic selenium and a metabolite may be dimethyl selenide.

The compound may be converted to a metabolite by oxidative degradation. In such embodiments, the compound may be converted to a metabolite in a non-enzymatic process. By “non-enzymatic process” is meant that the exogenous compound is not a substrate for an enzyme and instead the compound is broken down, or otherwise converted to the metabolite, by some other means. A compound may be an unsaturated fatty acid. A metabolite may be an alkene, for example a C2-C20 alkene, for example a C2-C10 alkene, for example a C4-C8 alkene, for example a C6 alkene, or for example 4-methyl-1-pentene and/or 1-hexene. Elevated breath levels of alkenes may result from lipid peroxidation of unsaturated fatty acids as a consequence of persistent inflammation and oxidative stress affecting a diseased liver, such as a cirrhotic liver.

### Compositions and Kits

The invention relates to a kit for the detection, diagnosis, screening or prognosis of a liver disease or for determining efficacy of a treatment for liver disease comprising one or more compound selected from tryptophan, a C1-C20 alkylbenzene, organic selenium, inorganic selenium and an unsaturated fatty acid and a device for capturing a biological sample from a patient.

In another embodiment, the kit may comprise:
i) one or more compound selected from limonene and eucolyptol; and
ii) one or more compound selected from 2-pentanone, tryptophan, a C1-C20 alkylbenzene, organic selenium, inorganic selenium and an unsaturated fatty acid;
and a device for capturing a biological sample from a patient.

The kit as described herein may include a composition for administration that comprises the compounds. This may be formulated as an oral administration, e.g., as a tablet or capsule.

The kit may comprise instructions for evaluating or monitoring liver disease in a patient based on the levels of the compounds and/or metabolites of interest. In some embodiments, the kit contains reagents for measuring the levels of the compounds and/or metabolites of interest.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed (e.g., sterile, pharmaceutically acceptable buffer and/or other diluents). However, various combinations of components may be comprised in a vial. The kits of the present invention also will typically include a means for containing the nucleic acids, and any other reagent containers in close confinement for commercial sale. Such containers may include injection or blow molded plastic containers into which the desired vials are retained.

This component of the kit may be for administration as described above. It may also include a pharmaceutically acceptable carrier or vehicle. This can be a particulate, so that the compositions are, for example, in tablet or powder form. The term “carrier” refers to a diluent, adjuvant or excipient, with which a substrate is administered. Such pharmaceutical carriers can be liquids, such as water and oils, including those of petroleum, animal, vegetable or synthetic origin, such as peanut oil, soybean oil, mineral oil, sesame oil and the like. The carriers can be saline, gum acacia, gelatin, starch paste, talc, keratin, colloidal silica, urea, and the like. In addition, auxiliary, stabilizing, thickening, lubricating and coloring agents can be used. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents. The composition can be in the form of a liquid, e.g., a solution, emulsion or suspension. The liquid can be useful for delivery by injection, infusion (e.g., IV infusion) or sub-cutaneously. As a solid composition for oral administration, the composition can be formulated into a powder, granule, compressed tablet, pill, capsule, chewing gum, wafer or the like form. Compositions can take the form of one or more dosage units.

Each compound can be contained in a composition, such as a nutritional supplement. The different absorption rates of the substrate into the blood can cause significant shifts in the time of maximum concentration on breath. Therefore, in one embodiment, compounds are provided in a formulation to ensure fast delivery. In one embodiment, the compounds are formulated as a liquid. In another embodiment, the compounds are formulated as a fast release/fast dissolving tablet or capsule. This ensures that the absorption has a much shorter time constant compared to the washout.

In another embodiment, the subject may be fasting overnight and fasting can be combined with the provision of the substrate as a liquid or fast release/dissolving tablet or fast release/dissolving capsule or other oral administration format.

Typically, the amount of a compound administered as part of the methods of the invention or the amount of a substrate included in the composition comprised in the kit is at least about 0.01% of the substrate by weight of the composition. When intended for oral administration, this amount can be varied to range from about 0.1% to about 80% by weight of the composition. For administration by injection, the composition can comprise from about typically about 0.1 mg/kg to about 250 mg/kg of the subject's body weight, preferably, between about 0.1 mg/kg and about 20 mg/kg of the subject's body weight, and more preferably from about 1 mg/kg to about 10 mg/kg of the subject's body weight.

### Uses

The invention also relates to the use of one or more exogenous compounds selected from tryptophan, a C1-C20 alkylbenzene, organic selenium, inorganic selenium and/or an unsaturated fatty acid in a method according to the fourth aspect of the invention.

The invention also relates to the use of two or more exogenous compounds selected from i) one or more of eucalyptol and/or limonene and ii) one or more of 2-pentanone, tryptophan, a C1-C20 alkylbenzene, organic selenium, inorganic selenium and/or an unsaturated fatty acid, in a method according to the fifth aspect of the invention.

Unless otherwise defined herein, scientific and technical terms used in connection with the present disclosure shall have the meanings that are commonly understood by those of ordinary skill in the art. While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

All documents mentioned in this specification are incorporated herein by reference in their entirety.

As used herein, unless otherwise expressly specified, all numbers such as those expressing values, ranges, amounts or percentages may be read as if prefaced by the word “about”, even if the term does not expressly appear. Also, the recitation of numerical ranges by endpoints includes all integer numbers and, where appropriate, fractions subsumed within that range (e.g. 1 to 5 can include 1, 2, 3, 4 when referring to, for example, a number of elements, and can also include 1.5, 2, 2.75 and 3.80, when referring to, for example, measurements). The recitation of end points also includes the end point values themselves (e.g. from 1.0 to 5.0 includes both 1.0 and 5.0). Any numerical range recited herein is intended to include all sub-ranges subsumed therein.

The terms “comprising”, “comprises” and “comprised of” as used herein are synonymous with “including”, “includes” or “containing”, “contains”, and are inclusive or open-ended and do not exclude additional, non-recited members, elements or method steps.

As used herein, the term “and/or is to be taken as specific disclosure of each of the two specified features or components with or without the other. For example, “A and/or B” is to be taken as specific disclosure of each of (i) A, (ii) B and (iii) A and B, just as if each is set out individually herein. Unless context dictates otherwise, the descriptions and definitions of the features set out above are not limited to any particular aspect or embodiment of the invention and apply equally to all aspects and embodiments which are described.

### EXAMPLES

### Methods

### Study Design and Subjects

A total of 44 subjects with cirrhosis, and 42 controls were enrolled with a random recruitment. Subjects >30 years of age were recruited from the clinical research facility at Addenbrooke's Hospital (Cambridge) or through the Cambridge BioResource. Patients had an established histological or radiological diagnosis of cirrhosis according to EASL and AASLD guidelines (European Association For The Study Of The L, European Organisation For R, Treatment Of C. EASL-EORTC clinical practice guidelines: management of hepatocellular carcinoma. J Hepatol 2012; 56:908-943; European Association for the Study of the Liver. Electronic address eee, European Association for the Study of the L. EASL Clinical Practice Guidelines: Management of hepatocellular carcinoma. J Hepatol 2018; 69:182-236). Disease severity was classified using the Child-Pugh (CP) scoring system. Only patients with CP class A or B were eligible for this study regardless of disease aetiology. Patients who developed hepatocellular carcinoma (HCC) in a cirrhosis background were not receiving any anti-cancer treatment at the time of sampling.

Control subjects had no known liver disease and were excluded if they were under investigation or had an history of malignancy in the last 2 years.

No dietary restrictions were applied to any participant.

### Breath Sampling

Acquisition of Breath Biopsy samples was performed in a single room at Addenbrooke's Hospital (Cambridge, UK) for all the subjects between August 2019 and March 2020. Breath samples were collected by adsorption onto Breath Biopsy Cartridges (made of four ¼″×3½″ inert-coated stainless-steel tubes with Tenax TA/carbograph 5TD adsorbent material (Markes International Ltd, Llantrisant, UK)) through a ReCIVA® Breath Sampler (Owlstone Medical, Cambridge, UK; Markar S R, Brodie B, Chin S T, Romano A, Spalding D, Hanna G B. Profile of exhaled-breath volatile organic compounds to diagnose pancreatic cancer. Br J Surg 2018; 105:1493-1500).

Prior to use for sampling, tubes were conditioned in a TC-20 (Markes International, Llantrisant, UK) by a N₂ flow at 20 psi and 320 °C for 4 hours. Before sampling, the ReCIVA was allowed to calibrate and adjust to the breathing pattern of the subject. 1.5 L of breath was sampled per tube at 225 mL/min. Ambient contamination was minimized by using the CASPER™® Portable Air Supply (Owlstone Medical, Cambridge, UK). Tubes were stored at a temperature of 4-8 °C for no more than 4 weeks before analysis. The same procedure was used for blanks collection, which was performed in the same room by applying the ReCIVA® Breath Sampler to a glass schott bottle.

### Analytical Measurements

Breath samples were analysed using Breath Biopsy OMNI global VOC analysis. Tubes containing breath samples were purged in a TD-100 (Markes International, Llantrisant, UK). Samples were first desorbed at 210 °C and focused onto a cold trap U-T12ME-2S, Material/Emission, C4-C32 (Markes international Ltd, Llantrisant, UK) at 20 °C. Focussed analytes were then desorbed at 300 °C for 3 minutes and purged by helium into the GC column (TraceGOLD TG-624SiIMS Thermo Fisher Scientific, US), with a sample flow split ratio of 10:1 and temperature gradient steps of 40 °C for 1 minute, 270 °C with a rate of 10 °C/min, and 300 °C with a rate of 30 °C/min for 5 minutes. Mass detection was performed using a Q Exactive™ GC Hybrid Quadrupole-Orbitrap™ Mass Spectrometer (Thermo Fisher™ Scientific Inc, US) scanning from m/z 30 to m/z 450 with a resolving power of 60,000. Electron ionization voltage was set at 70 eV. The ion source and heater temperatures were respectively fixed at 250 °C and 230 °C. Before every sequence, the system was leak checked, tuned, and calibrated.

### Feature Extraction

Raw data files, collected in profile mode, were centroided using the peak picking Vendor algorithm of the ProteoWizard-MSConvert application (Chambers M C, Maclean B, Burke R, Amodei D, Ruderman D L, Neumann S, et al. A cross-platform toolkit for mass spectrometry and proteomics. Nat Biotechnol 2012; 30:918-920). The centroid data were imported into MZmine 2.53 to proceed with feature extraction workflow (Pluskal T, Castillo S, Villar-Briones A, Oresic M. MZmine 2: modular framework for processing, visualizing, and analyzing mass spectrometry-based molecular profile data. BMC Bioinformatics 2010; 11:395). ADAP chromatogram builder module was applied to detect the peak with the following parameters (minimum highest intensity: 1.0E5; m/z tolerance: 5 PPM; Myers O D, Sumner S J, Li S, Barnes S, Du X. One Step Forward for Reducing False Positive and False Negative Compound Identifications from Mass Spectrometry Metabolomics Data: New Algorithms for Constructing Extracted lon Chromatograms and Detecting Chromatographic Peaks. Anal Chem 2017; 89:8696-8703). Detected features were deconvoluted using Wavelet ADAP algorithm with the following parameters (S/N: 3; S/N estimator: wavelet coeff.SN, coefficient/area threshold: 100, peak duration range: 0-1.0 minutes, RT wavelet range, 0-0.15 minutes). Hierarchical clustering was used to combine peaks into analytes and construct fragmentation spectra for each analyte as described by Du X, et.al (Smirnov A, Jia W, Walker D I, Jones D P, Du X. ADAP-GC 3.2: Graphical Software Tool for Efficient Spectral Deconvolution of Gas Chromatography-High-Resolution Mass Spectrometry Metabolomics Data. J Proteome Res 2018; 17:470-478). ADAP Aligner (GC) was used for retention time alignment, minimum confidence: 0.1, RT tolerance: 0.5 minutes, m/z tolerance: 5 ppm, score threshold: 0.75, score weight 0.1, retention time similarity was calculated using Retention Time Difference (fast) method). Compounds were tentatively identified using the National Institute of Standards and Technology (NIST) library and in-house High Resolution Accurate Mass (HRAM) library (https://www.owlstonemedical.com/about/blog/2022/may/30/biomarker-analysis-breath-hram-library/). Database hits were filtered according to their structure relevance to standard compounds. Identity of compounds was validated by standards injection ((R)-(+)-Limonene: product ref. 183164 from SigmaAldrich; 2-pentanone: product ref. 471194 from SigmaAldrich; dimethyl selenide: product ref. 41572 from SigmaAldrich; Indole: product ref. 13408 from SigmaAldrich; and Eucalyptol: product ref. 29210 from SigmaAldrich).

### Data Handling and Statistical Analysis

Data were analysed by using both Python and R programming languages (Python Software Foundation. Python Language Reference, version 2.7); R Core Team (2021). R: A language and environment for statistical computing. R Foundation for Statistical Computing, Vienna, Austria). Data visualization was performed using libraries matplotlib, seaborn, and ggplot2.

Intensity of VOCs (count/min) median and range were compared between groups using the Mann-Whitney U-test for non-parametric data. A Mann-Whitney U-test p-value <0.05, was considered statistically significant (unless differently stated). A total of 11 VOCs showing a Benjamin-Hochberg adjusted p-value <0.1 advanced to investigation for correlation with blood metrics of liver function. Data was pre-processed as follows: breath sample technical duplicates were averaged. Correction for instrument fluctuation was obtained by Probabilistic Quotient Normalisation (PQN). Log transformation was performed to bring VOCs intensity closer to the normal distribution.

To build a classification model, first dimensionality reduction was performed by considering as candidate biomarkers only features identified as statistically significant in the univariate analysis (p<0.05) between cirrhosis and controls. This selection reduces the feature space that any model needs to search, and thus likelihood of unstable results or overfitting. Top VOCs for classification were identified by using stability selection with LASSO Logistic Regression. The model's performance was validated with 5-fold cross validation, with VOC selection performed within the training set of each fold, and only features above a certain stability score are selected in the model. The importance of each feature for classification can then be expressed as the resulting average stability score across the 5-fold cross validation.

While stability selection chooses as many features as it can that behave consistently through permutations of a dataset, in the context of a clinical test, it is sometimes important to reduce the number of features/VOCs required for measurement further. To this end, an analysis was conducted to observe with how few features a similar ROC-AUC could be obtained compared to the stable model. Step-forward feature selection, with a simple 5-fold cross validation was applied to obtain the best performing model with only one feature, only two features, and so on, minimizing the number of VOCs necessary for a potential breath test. Starting from a feature set size of one, classification performance of combinations of candidate biomarkers expressed as ROC-AUC was measured by adding one new VOC at a time to the model.

Within the cirrhosis group, the relationship between blood bilirubin, albumin and INR, and breath metrics was assessed by using canonical correlation analysis (CCA). CCA finds the relationship between two multivariate sets of variables measured for the same set of samples and is considered as the extension of bivariate correlations. The identified new bases (new directions) for each data set are a linear combination of the original parameters. The new bases (called canonical variates) represent maximized correlations between the original parameters in two datasets (i.e., VOCs and Blood metrics). The resulting CCA score plot is generated by using the statistically significant canonical variates. In the score plot, each point corresponds to the combined information from breath and blood sample collected from the same patients. The shape of the projected data point indicates the correlation between two blocks of data (here VOCs in breath and blood metrics). The contribution of the original parameters to the correlation between the two blocks of variables can be estimated by calculating the canonical loadings, which express the correlation between the original variable and the canonical variate. Variance of selected features in relation to severity of cirrhosis was assessed by principal component analysis (PCA).

### Results

Identification of VOCs Associated with Cirrhosis

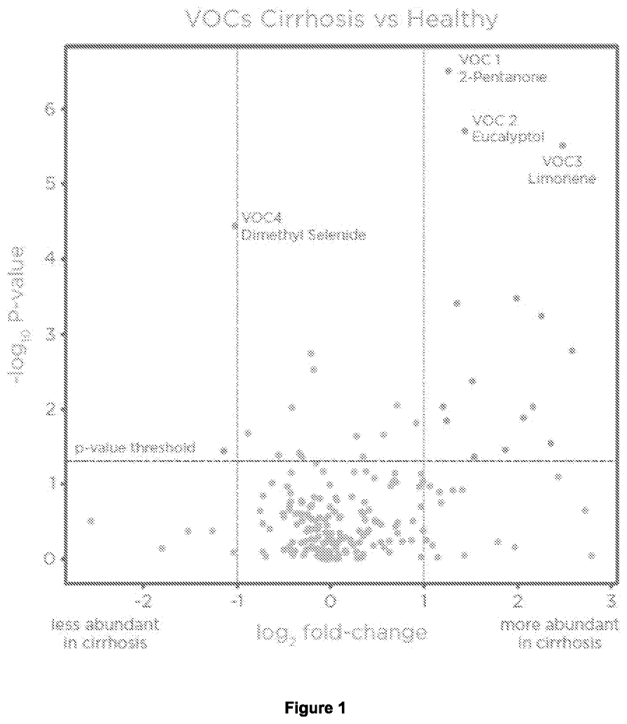

Abundance of signals from features measured in the breath of subjects with cirrhosis compared to controls was compared by using univariate analysis. As shown in FIG. 1, a subset of breath VOCs were found elevated, while some were found reduced in the breath of subjects with cirrhosis compared to controls. VOCs with a median fold change >2, and a p-value <0.05 (Unadjusted Mann-Whitney U-test) are highlighted in FIG. 1.

Limonene (m/z 91.05 and RT of ~11 min), eucalyptol and 2-pentanone (m/z 71.04 and RT of ~5 min) were found upregulated in the breath of patients with cirrhosis. Dimethyl selenide (m/z 109.96 and RT of ~3 min) was found downregulated in the breath of patients with cirrhosis.

### Estimation of Classification Performance

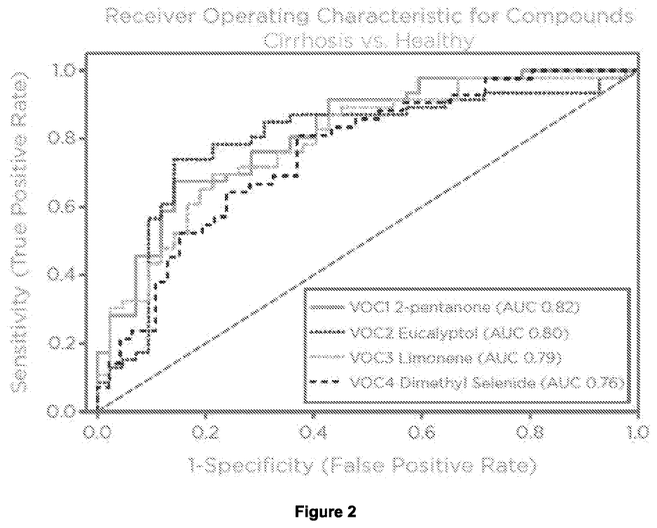

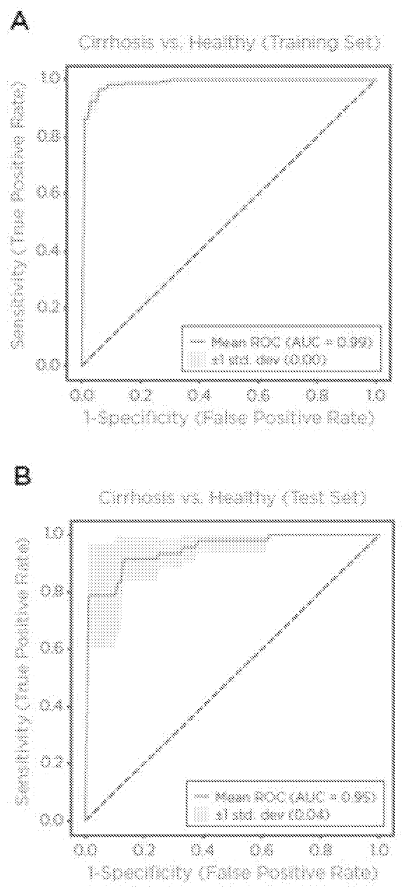

A total of 29 on-breath VOCs were differentially abundant (unadjusted p-value <0.05, Mann-Whitney U-test) between healthy and cirrhosis groups and were first used to generate individual receiving operator characteristic (ROC) plots to estimate individual diagnostic performance predicting the presence of cirrhosis. The top 4 compounds: 2-pentanone, eucalyptol, limonene and dimethyl selenide, with area under the ROC curve (ROC-AUC) of 0.82, 0.80, 0.79, and 0.76, respectively, are shown in FIG. 2.

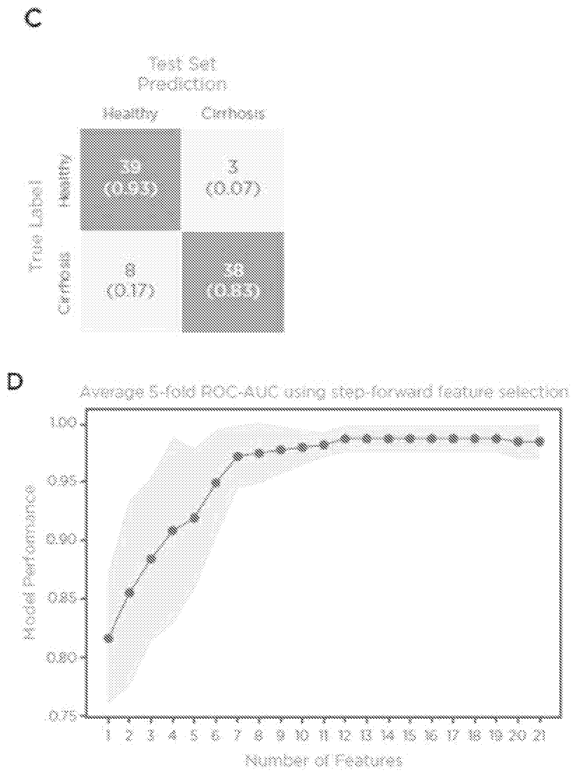

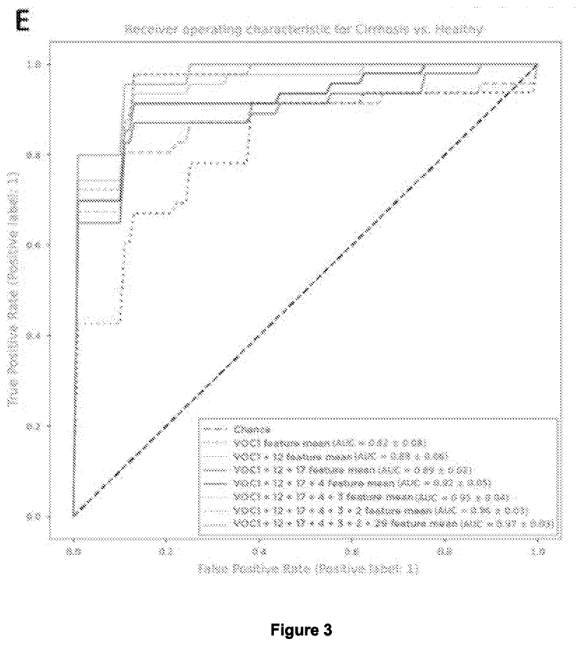

Subsequently, the performance of combinations of VOCs was explored. A stratified 5-fold cross validation train/test split (70%/30%) was performed to build a classification model. On the training sets, the model returned an average ROC-AUC of 0.99±0.00 (FIG. 3A), and on the test sets, an average ROC-AUC of 0.95±0.04 (FIG. 3B). The corresponding confusion matrix, shown in FIG. 3C, generated by cross-validated predictions shows 3 false positives (7%), who were misclassified because of elevated levels of limonene or 2-pentanone. Of the 8 false negative (17%), 6 were CP class A and 2 were CP class B.

Step forward feature selection was used to identify the most important VOCs for classification. This method adds one feature at the time to the classification model to establish how much classification performance improves. As shown in FIGS. 3 D-E, the first 7 features are those contributing the most to the classification performance. Identity of these top performing 7 VOCs is reported in Table 1.

The results show that the relative amounts of each of 2-pentanone, 4-methyl-1-pentene and/or 1-hexene, indole, dimethyl selenide, limonene, eucalyptol, and 1-propylnonyl)benzene are required to detect, stage, monitor or prognose a liver disease in an untargeted method.

TABLE 1
Identity of top performing VOCs

| | | | | Average | |
|---|---|---|---|---|---|
| | Compound | | | stability | Identifi- |
| VOC # | ID | RT | m/z | score | cation |
| VOC1 | 2-Pentanone | 5.30598 | 71.04919 | 0.916 | HRAM* |
| | | | | | Standard |
| VOC12 | 4-methyl- | 7.62411 | 42.03163 | 0.890 | NIST |
| | 1-pentene | | | | |
| | or 1-hexene | | | | |
| VOC17 | Indole | 17.13762 | 116.90607 | 0.886 | HRAM |
| | | | | | Standard |
| VOC4 | Dimethyl | 3.30723 | 109.963 | 0.762 | HRAM |
| | selenide | | | | Standard |
| VOC3 | Limonene | 11.00585 | 91.05427 | 0.734 | HRAM |
| | | | | | Standard |
| VOC2 | Eucalyptol | 11.34312 | 139.11181 | 0.732 | HRAM |
| | | | | | Standard |
| VOC29 | (1-propyl- | 20.9004 | 133.19707 | 0.6 | NIST |
| | nonyl)benzene | | | | |

Identification of Breath Compounds Correlated with Hepatic Function in Subjects with Cirrhosis

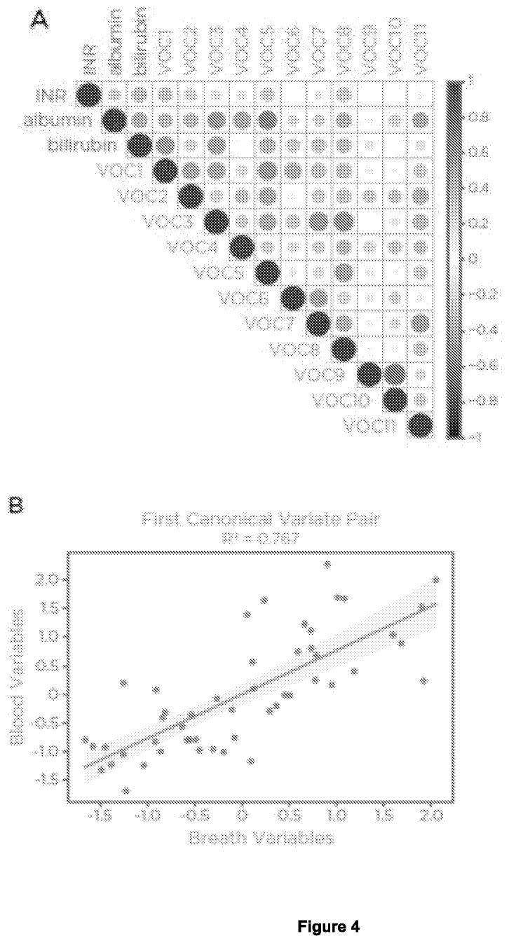

Correlations between selected breath compounds and blood metrics of liver function within the cirrhosis group, namely bilirubin, albumin, and prothrombin time expressed as international normalized ratio (INR), were first investigated by generating a Pearson correlation matrix (FIG. 4A). Red indicates a positive correlation; blue indicates a negative correlation.

Collective correlations between breath and blood metrics of subjects with cirrhosis were further investigated by using the canonical correlation analysis (CCA). CCA finds the relationship between two multivariate sets of variables measured for the same set of samples and is considered as the extension of bivariate correlations. The identified new bases (new directions) for each data set are a linear combination of the original parameters. The new bases (called canonical variates) represent maximized correlations between the original parameters in two datasets (i.e., VOCs and Blood metrics). The resulting CCA score plot is generated by using the statistically significant canonical variates. In the score plot, each point corresponds to the combined information from breath and blood sample collected from the same patients. The shape of the projected data point indicates the correlation between two blocks of data (here VOCs in breath and blood metrics). As shown in FIG. 4B, the resulting CCA score plot of the first canonical variates showed that the set of variables measured in blood significantly correlate with the set of variables measured in breath (R2=0.767).

### Example 5

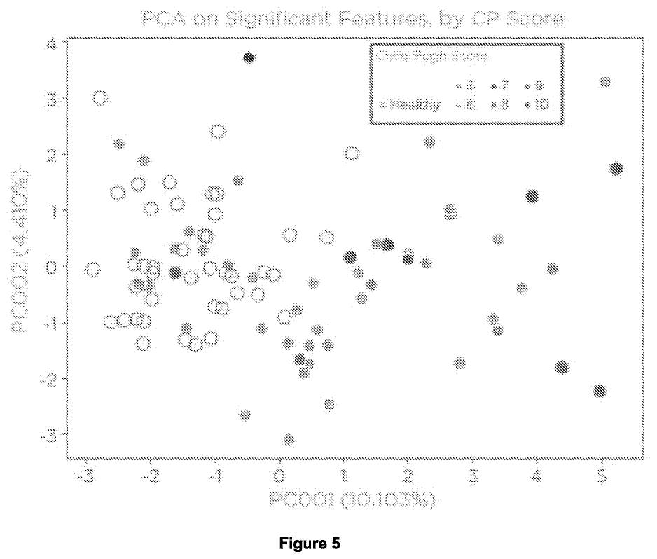

The relation between the identified VOCs and the Child-Pugh (CP) score scoring system, which is based on the levels of bilirubin and albumin and the international normalized ratio (INR), was investigated as a readout of hepatic function impairment associated with cirrhosis. The subset of VOCs that correlated with blood metrics were used to perform a principal component analysis (PCA) and projected data points for each patient were coloured by CP score as shown in FIG. 5. PC1 explained 10.1% and PC2 4.4% of variance and showed mainly a separation of patients with CP score >5 from healthy controls.

1. A method for detecting, staging, monitoring or prognosing a liver disease in a subject, the method comprising measuring the concentration of each of the following compounds in a biological sample obtained from the subject: i) 2-pentanone, ii) 4-methyl-1-pentene and/or 1-hexene, iii) indole, iv) dimethyl selenide, v) limonene, vi) eucalyptol and vii) (1-propylnonyl)benzene.

2. The method according to claim 1, wherein the method comprises:
(a) establishing a set of test subject values based on the concentration of each of the following compounds i) to vii) in the biological sample obtained from the subject; and
(b) comparing the set of test subject values to a set of reference values, wherein a difference in the set of test subject values and the set of reference values indicates a likelihood of a liver disease.

3. The method according to any of claim 1 or 2, wherein the subject has not been administered with an exogenous substrate for an enzyme associated with a liver disease.

4. The method according to any preceding claim, wherein the set of reference values are based on the concentration of each of compounds i) to vii) in a biological sample obtained from a subject that has been diagnosed with a liver disease.

5. The method according to any of claims 1-3, wherein the set of reference values are based on the concentration of each of compounds i) to vii) obtained from a biological sample obtained from a healthy subject.

6. The method according to claim 5, wherein the concentration of each of 2-pentanone, 4-methyl-1-pentene and/or 1-hexene, indole, limonene, eucalyptol and propylnonyl)benzene in a biological sample obtained from the subject are elevated compared to the concentration of said compounds in a biological sample obtained from a healthy subject.

7. The method according to any one of claim 5 or 6, wherein the concentration of dimethyl selenide in a biological sample obtained from the subject is reduced compared to the concentration of indole in a biological sample obtained from a healthy subject.

8. The method according to any preceding claim, wherein the biological sample is selected from breath, urine, blood, serum, and/or tissue.

9. The method according to claim 8, wherein the biological sample is exhaled breath.

10. The method according to claim 9, wherein the method comprises i) the subject inhaling purified air and ii) the subject providing an exhaled breath sample, and wherein step i) is carried out before step ii).

11. The method according to any preceding claim, wherein the liver disease is a liver disease with cirrhosis.

12. The method according to any preceding claim, wherein the liver disease is non-alcoholic steatohepatitis (NASH).

13. The method according to any preceding claim, wherein the liver disease is NASH without fibrosis, NASH with fibrosis, NASH with hepatocellular carcinoma (HCC) or NASH with cirrhosis.

14. The method according to claim 13, wherein the liver disease is NASH with decompensated cirrhosis.

15. The method according to any preceding claim, further comprising the step of treating the subject.

16. A method for determining efficacy of a treatment in a subject diagnosed with a liver disease, comprising the steps of:
(a) obtaining a first biological sample from the patient before initiation of the treatment or therapy (or at a first time point after initiation of the treatment or therapy, or when the treatment or therapy is initiated);
(b) determining the concentration of each of compounds i) to vii) in the first sample;
(c) obtaining a second biological sample from the patient after initiation of the treatment or therapy (or at a second time point after initiation of the treatment or therapy);
(d) determining the concentration of each of compounds i) to vii) in the second sample; and
(e) comparing the concentration of each of compounds i) to vii) in the first sample with the concentration of each of compounds i) to vii) in the second sample.

17. A method for detecting, staging, monitoring or prognosing liver disease in a subject, comprising measuring the concentration of an exogenous compound and/or metabolite thereof in a biological sample obtained from said subject; wherein the exogenous compound is a substrate for an enzyme, which substrate may be metabolised to the metabolite by said enzyme in vivo, and/or is a compound that may undergo oxidative degradation in vivo to form the metabolite; wherein the exogenous compound is a generally recognised as safe (GRAS) compound; and wherein the exogenous compound is selected from tryptophan, a C1-C20 alkylbenzene, organic selenium, inorganic selenium and/or an unsaturated fatty acid.

18. A method for detecting, staging, monitoring or prognosing liver disease in a subject, comprising measuring the concentration of two or more exogenous compounds and/or metabolites thereof in a biological sample obtained from said subject; wherein each exogenous compound is a substrate for an enzyme, which substrate may be metabolised to the metabolite by said enzyme in vivo, and/or is a compound that may undergo oxidative degradation in vivo to form the metabolite; wherein each exogenous compound is a generally recognised as safe (GRAS) compound; and wherein the exogenous compounds are selected from:
i) one or more of eucalyptol and/or limonene; and
ii) one or more of 2-pentanone, tryptophan, a C1-C20 alkylbenzene, such as (1-propylnonyl)benzene, organic selenium, inorganic selenium and/or an unsaturated fatty acid.

19. A method according to claim 18, wherein the method comprises measuring the concentration of each of the following exogenous compounds and/or metabolites thereof: I) eucalyptol, II) limonene, III) 2-pentanone, IV) tryptophan, V) a C1-C20 alkylbenzene, VI) organic selenium, VII) inorganic selenium and VIII) an unsaturated fatty acid.

20. A method according to claim 19, wherein the C1-C20 alkylbenzene is (1-propylnonyl)benzene.

21. A method according to any of claims 17-20, wherein the metabolite of tryptophan comprises indole.

22. A method according to any of claims 17-21, wherein the metabolite of a C1-C20 alkylbenzene comprises benzoic acid and/or hippuric acid.

23. A method according to any of claims 17-22, wherein the metabolite of organic selenium and/or inorganic selenium comprises dimethyl selenide.

24. A method according to any of claims 17-23, wherein the metabolite of an unsaturated fatty acid comprises an alkene.

25. A method according to claim 24, wherein the alkene is a C2-C20 alkene.

26. A method according to claim 24, wherein in the alkene is a C2-C10 alkene,

27. A method according to claim 24, wherein the alkene is a C4-C8 alkene.

28. A method according to claim 24, wherein the alkene is a C6 alkene.

29. A method according to claim 24, wherein the alkene is 4-methyl-1-pentene and/or 1-hexene.

30. A method according to any of claims 18-29, wherein the metabolite of eucalyptol comprises 2-alpha-hydroxy-1,8-cineole and/or 3-alpha-hydroxy-1,8-cineole.

31. A method according to any of claims 18-30, wherein the metabolite of limonene comprises trans-carveol and/or perillyl alcohol.

32. A method according to any of claims 18-31, wherein the metabolite of 2-pentanone comprises 2-pentanol, 3-hydroxy-2-pentanone and/or 2,3-pentanediol.

33. A method according to claim 32, wherein the metabolite of 2-pentanone comprises 2-pentanol.

34. Use of one or more exogenous compounds selected from tryptophan, a C1-C20 alkylbenzene, organic selenium, inorganic selenium and/or an unsaturated fatty acid in a method according to claim 17.

35. Use of two or more exogenous compounds selected from i) one or more of eucalyptol and/or limonene and ii) one or more of 2-pentanone, tryptophan, a C1-C20 alkylbenzene, organic selenium, inorganic selenium and/or an unsaturated fatty acid, in a method according to claim 18.

36. A method for detecting, staging, monitoring or prognosing liver disease in a subject, comprising measuring the concentration of each of the following exogenous compounds and/or metabolites thereof: I) eucalyptol, II) limonene, III) 2-pentanone, IV) tryptophan, V) a C1-C20 alkylbenzene, VI) organic selenium, VII) inorganic selenium and VIII) an unsaturated fatty acid in a biological sample obtained from said subject.

37. A method according to claim 36, wherein the C1-C20 alkylbenzene is (1-propylnonyl)benzene.

38. A method according to any of claims 36-37, wherein the metabolite of tryptophan comprises indole.

39. A method according to any of claims 36-38, wherein the metabolite of a C1-C20 alkylbenzene comprises benzoic acid and/or hippuric acid.

40. A method according to any of claims 36-39, wherein the metabolite of organic selenium and/or inorganic selenium comprises dimethyl selenide.

41. A method according to any of claims 36-40, wherein the metabolite of an unsaturated fatty acid comprises an alkene.

42. A method according to claim 41, wherein the alkene is a C2-C20 alkene.

43. A method according to claim 41, wherein in the alkene is a C2-C10 alkene,

44. A method according to claim 41, wherein the alkene is a C4-C8 alkene.

45. A method according to claim 41, wherein the alkene is a C6 alkene.

46. A method according to claim 41, wherein the alkene is 4-methyl-1-pentene and/or 1-hexene.

47. A method according to any of claims 36-46, wherein the metabolite of eucalyptol comprises 2-alpha-hydroxy-1,8-cineole and/or 3-alpha-hydroxy-1,8-cineole.

48. A method according to any of claims 36-47, wherein the metabolite of limonene comprises trans-carveol and/or perillyl alcohol.

49. A method according to any of claims 36-48, wherein the metabolite of 2-pentanone comprises 2-pentanol, 3-hydroxy-2-pentanone and/or 2,3-pentanediol.

50. A method according to claim 49, wherein the metabolite of 2-pentanone comprises 2-pentanol.

51. Use of each of I) eucalyptol, II) limonene, III) 2-pentanone, IV) tryptophan, V) a C1-C20 alkylbenzene, VI) organic selenium, VII) inorganic selenium and VIII) an unsaturated fatty acid in a method according to claim 36.
